(19) United States
(12) Patent Application Publication
(10) Pub. No.: US 2024/0366954 A1
(43) Pub. Date: Nov. 7, 2024

(54) COMPOSITIONS, SYSTEMS, AND METHODS FOR TREATING CANCER USING TUMOR TREATING FIELDS AND KILLER CELLS

(71) Applicant: Novocure GmbH, Root D4 (CH)

(72) Inventors: Tal KAN, Haifa (IL); Yiftah BARSHESHET, Haifa (IL)

(73) Assignee: Novocure GmbH, Root D4 (CH)

(21) Appl. No.: 18/619,957

(22) Filed: Mar. 28, 2024

Related U.S. Application Data
(60) Provisional application No. 63/493,141, filed on Mar. 30, 2023.
(60) Provisional application No. 63/599,142, filed on Nov. 15, 2023.

(51) Int. Cl. A61N 1/40; A61K 35/17; A61K 39/00

(52) U.S. Cl. CPC A61N 1/40; A61K 35/17; A61K 39/4613; A61K 39/4631

(57) ABSTRACT

Compositions, systems, and methods for reducing viability of cancer cells and treating cancer, as well as preventing an increase of volume of a tumor present in a body of a living subject, are disclosed. The systems and methods involve application of an alternating field concurrently with administration of at least one composition comprising at least one killer cell (such as, but not limited to, at least one natural killer (NK) cell or at least one cytokine-induced killer (CIK) cell).

### REFERENCE TO RELATED APPLICATIONS

The subject application claims benefit under 35 USC § 119(e) of U.S. Provisional Application No. 63/493,141, filed Mar. 30, 2023; and U.S. Provisional Application No. 63/599,142, filed Nov. 15, 2023. The entire contents of the above-referenced patent applications are hereby expressly incorporated herein by reference.

### STATEMENT REGARDING FEDERALLY SPONSORED RESEARCH

Not Applicable.

### BACKGROUND ART

Tumor Treating Fields (TTFields) are low intensity (e.g., 1-3 V/cm) alternating electric fields within the intermediate frequency range (such as, but not limited to, 100-500 kHz) that target solid tumors by disrupting mitosis. This non-invasive treatment targets solid tumors and is described, for example, in U.S. Pat. Nos. 7,016,725; 7,089,054; 7,333,852; 7,565,205; 8,244,345; 8,715,203; 8,764,675; 10,188,851; and 10,441,776. TTFields are typically delivered through two pairs of transducer arrays that generate perpendicular fields within the treated tumor; the electrode arrays that make up each of these pairs are positioned on opposite sides of the body part that is being treated. More specifically, for the OPTUNE® system, one pair of electrodes is located to the left and right (LR) of the tumor, and the other pair of electrodes is located anterior and posterior (AP) to the tumor. TTFields are approved for the treatment of glioblastoma multiforme (GBM), and may be delivered, for example, via the OPTUNE® system (Novocure Limited, St. Helier, Jersey), which includes transducer arrays placed on the patient's shaved head.

Each transducer array used for the delivery of TTFields in the OPTUNE® device comprises a set of ceramic disk electrodes, which are coupled to the patient's skin (such as, but not limited to, the patient's shaved head for treatment of GBM) through a layer of conductive medical gel. The purpose of the medical gel is to deform to match the body's contours and to provide good electrical contact between the arrays and the skin; as such, the gel interface bridges the skin and reduces interference. The device is intended to be continuously worn by the patient for 2-4 days before removal for hygienic care and re-shaving (if necessary), followed by reapplication with a new set of arrays. As such, the medical gel remains in substantially continuous contact with an area of the patient's skin for a period of 2-4 days at a time. In addition, the arrays can be shifted a few centimeters in either direction to allow the skin to heal from one period of treatment to the next. Therefore, a portion of skin that was covered by electrodes/gel for a 2-4 day period could then be uncovered for 2-4 days when the replaced electrodes are shifted slightly; then the device may be reapplied to the original portion of skin for the next 2-4 day period.

Natural killer (NK) cells are a type of innate lymphoid cell population. NK cells can differentiate between healthy (i.e., self) and infected or abnormal cells, including transformed cancer cells. Unlike T and B cells, NK cells are capable of attacking and killing abnormal cells (including cancer cells) in an antigen-independent manner (Shimasaki et al. (2020) Nat Rev Drug Discov, 19:200-218)). Healthy cells express low levels of NK-cell-activating ligands and high levels of major histocompatibility (MHC) class I (in humans HLA—human leukocyte antigen). MHC Class I binds killer immunoglobulin-like (KIR) receptors on NK cells and prevents the NK cell from killing the healthy cell (Liu et al. (2021) J Hematol Oncol, 14:7)). Downregulation of MHC Class I in infected or cancerous cells can lead to NK cell activation and lysing of the transformed cell (Liu et al., 2021). NK cells have other stimulatory and inhibitory signals that are facilitated via receptor-ligand interactions with the target cell. For example, HLA-E, a nonclassical HLA class I molecule binds NK cells via NKG2A receptor and acts as an inhibitory signal for NK cells (Lauterbach et al. (2015) Human Immunology, 76(8):578-586)). Accordingly, high HLA-E levels can be found in several cancer types, including non-small cell lung carcinoma (NSCLC), pancreas, kidney, melanoma, head and neck carcinoma, and more (Borst et al. (2020) Clinical Cancer Research, 26(21):5549-5556).

Most clinical trials using NK cell-based immunotherapies were in hematological malignancies. However, clinical trials of NK cells in solid tumors such as breast and ovarian cancer have also been conducted (Chu et al. (2022) J Transl Med, 20:240).

In addition, Silginer et al. (Neuro-Oncology (2018) 20(6):vi133) investigated the interaction of TTFields with molecules involved in immune responses as well as drugs that may modulate immune cell activity and found increased NKG2D ligand expression and enhanced NK cell-based killing of glioma cells exposed to TTFields while MHC Class I and Class II expression remained unaltered.

Cytokine-induced killer (CIK) cell therapy is a type of adoptive immunotherapy that uses a heterogeneous population of immune cells generated ex vivo from patient peripheral blood mononuclear cells (PBMCs) in the presence of IL-2, anti-CD3 antibody (such as, but not limited to, OKT3), and IFN-gamma (Introna (2017) Journal of Autoimmunity, 85:32-44). The CIK cells include a CD3⁺CD56⁺CD8⁺ population, which are phenotypically cytotoxic T-terminally differentiated effector memory (EMRA) lymphocytes (Franceschetti et al. (2009) Experimental Hematology, 37:616-628.e2) with antitumor activity. This population expresses different NK markers, including NKG2D, and is able to recognize and kill tumor cells in a TCR-independent, NK-like manner (Sangiolo et al. (2008) International Immunology, 20:841-848). According to a recent report from the International Registry on CIK Cells (IRCC), CIK cells have been tested in the setting of 106 clinical trials in over 30 distinct tumors, with a significant improvement in progression-free survival (PFS) and overall survival (OS) in 27 trials in the autologous setting, with mild adverse effects. CIK cells have also been evaluated in patients with GBM, the most common malignant primary brain tumor (Kong et al. (2017) Oncotarget, 8:7003-7013). In this phase III trial, CIK cells were associated with improvement in PFS but not OS. This trial is one of several immunotherapy trials with poor clinical outcomes in GBM, including immune checkpoint inhibitors, vaccinations, and different adoptive T cell therapies (Weenink et al. (2020) Cancers (Basel), 12:E751).

### BRIEF DESCRIPTION OF THE DRAWINGS

FIG. 1 graphically depicts an analysis of MHC Class I expression in human and mouse pancreatic cell lines (1A-AsPc1 human pancreatic cancer cell line and 1B-Panc02 mouse pancreatic cancer cell line) following TTFields exposures of 72 or 96 hours.

FIG. 2 graphically depicts transcriptomics analysis following TTFields treatment compared with control in glioblastoma (GBM) cell lines, malignant pleural mesothelioma cell line (MPM), and pancreatic cancer cell lines AsPC1 and BxPC3, to identify genes that exhibit a difference in expression following TTFields treatment.

FIG. 3 graphically depicts CIK cells expansion. CIK cells were generated in vitro from HD and counted at different time points. (a) Cells were counted at different time points. Data shown are the median and interquartile range of cell counts from 5 separate experiments. After 21 days (±1 day) of culture, CIK were characterized by flow cytometry for the expression of (b) CD3,C56 and (c) CD4 and CD8. The data shown are the mean of 5 separate experiments.

FIG. 4 graphically depicts CIK/glioblastoma stem cell (GSC) co-culture. After 24 hours or 48 hours of co-culture, GSC viability was evaluated by flow cytometry and plotted as the percentage of dead cells (L/D+) cells among cells labeled with the CellTrace. GSCs displayed a T/E ratio-dependent increase in the apoptotic fraction when cocultured with CIK cells (a) but not with PBMC (b). The results presented are the mean of duplicates of a single experiment.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference

All of the compositions, assemblies, systems, kits, and/or methods disclosed herein can be made and executed without undue experimentation in light of the present disclosure. While the compositions, assemblies, systems, kits, and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (e.g., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

The term “pharmaceutically acceptable” refers to compounds and compositions which are suitable for administration to humans and/or animals without undue adverse side effects such as (but not limited to) toxicity, irritation, and/or allergic response commensurate with a reasonable benefit/risk ratio.

The term “patient” or “subject” as used herein includes human and veterinary subjects. “Mammal” for purposes of treatment refers to any animal classified as a mammal, including (but not limited to) humans, domestic and farm animals, nonhuman primates, and any other animal that has mammary tissue.

The term “treatment” refers to both therapeutic treatment and prophylactic or preventative measures. Those in need of treatment include, but are not limited to, individuals already having a particular condition/disease/infection as well as individuals who are at risk of acquiring a particular condition/disease/infection (e.g., those needing prophylactic/preventative measures). The term “treating” refers to administering an agent/element/method to a patient for therapeutic and/or prophylactic/preventative purposes.

The term “therapeutic composition” or “pharmaceutical composition” as used herein refers to an agent that may be administered in vivo to bring about a therapeutic and/or prophylactic/preventative effect.

Administering a therapeutically effective amount or prophylactically effective amount is intended to provide a therapeutic benefit in the treatment, prevention, and/or management of a disease, condition, and/or infection. The specific amount that is therapeutically effective can be readily determined by the ordinary medical practitioner, and can vary depending on factors known in the art, such as (but not limited to) the type of condition/disease/infection, the patient's history and age, the stage of the condition/disease/infection, and the co-administration of other agents.

The term “effective amount” refers to an amount of a biologically active molecule or conjugate or derivative thereof, or an amount of a treatment protocol (e.g., an alternating electric field), sufficient to exhibit a detectable therapeutic effect without undue adverse side effects (such as (but not limited to) toxicity, irritation, and allergic response) commensurate with a reasonable benefit/risk ratio when used in the manner of the inventive concept(s). The therapeutic effect may include, for example but not by way of limitation, preventing, inhibiting, or reducing the occurrence of at least one condition, disease, and/or infection. The effective amount for a subject will depend upon the type of subject, the subject's size and health, the nature and severity of the condition/disease/infection to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “concurrent therapy” is used interchangeably with the terms “concomitant therapy” and “adjunct therapy,” and will be understood to mean that the patient in need of treatment is treated or given another drug for the condition/disease/infection in conjunction with the treatments of the present disclosure. This concurrent therapy can be sequential therapy, where the patient is treated first with one treatment protocol/pharmaceutical composition and then the other treatment protocol/pharmaceutical composition, or the two treatment protocols/pharmaceutical compositions are given simultaneously. In addition, it will be understood that one administration step (such as, but not limited to, administration of the TTFields) may occur over a longer period of time than the other administration step (i.e., oral administration or injection of a substance). In these instances of varying administration time periods, the term “simultaneously” will be understood to mean that the shorter administration step wholly overlaps with the longer administration step. However, the term “simultaneously” will include performing the shorter administration step at any point during the longer administration step (e.g., the beginning, middle, or end of the longer administration step, or any other time period therebetween), as well as performing the shorter administration step one or more times wholly within the time period of the longer administration step. Therefore, the term “simultaneously” does not require that the two administration steps be performed over the exact same length of time.

The terms “administration” and “administering,” as used herein, will be understood to include all routes of administration known in the art, including but not limited to, oral, topical, transdermal, parenteral, subcutaneous, intranasal, mucosal, intramuscular, intraperitoneal, intravitreal, and intravenous routes, and including both local and systemic applications. In addition, the compositions of the present disclosure (and/or the methods of administration of same) may be designed to provide delayed, controlled, or sustained release using formulation techniques which are well known in the art.

The term “target region,” as used herein, refers to a region containing all or a portion of the cancer, cancer cells, and/or tumor to be treated.

Turning now to the inventive concept(s), a concurrent therapy for cancer is disclosed herein. The concurrent therapy includes the use of alternating electric fields (e.g., TTFields) in addition to killer cells (such as, but not limited to, natural killer (NK) or cytokine-induced killer (CIK) cells). The combination of alternating electric fields (e.g., TTFields) with killer cells provides a synergistic result in the treatment of cancer.

Certain non-limiting embodiments of the present disclosure are directed to a method of reducing viability of cancer cells. The method includes the steps of: (1) applying an alternating electric field to the cancer cells for a period of time; and (2) administering at least one composition to the cancer cells, wherein the at least one composition comprises at least one killer cell (such as, but not limited to, at least one natural killer (NK) cell or at least one cytokine-induced killer (CIK) cell). Such methods may be carried out in vitro or in vivo.

Certain additional non-limiting embodiments of the present disclosure are directed to a method of treating cancer in a subject. The method includes the steps of: (1) applying an alternating electric field to a target region of the subject for a period of time; and (2) administering at least one composition to the subject, wherein the at least one composition comprises at least one killer cell (such as, but not limited to, at least one natural killer (NK) cell or at least one cytokine-induced killer (CIK) cell).

Certain additional non-limiting embodiments of the present disclosure are directed to a method of reducing a volume of a tumor present in a body of a living subject, wherein the tumor includes a plurality of cancer cells. The method includes the steps of: (1) applying an alternating electric field to a target region of the subject for a period of time; and (2) administering at least one composition to the subject, wherein the at least one composition comprises at least one killer cell (such as, but not limited to, at least one natural killer (NK) cell or at least one cytokine-induced killer (CIK) cell).

Certain additional non-limiting embodiments of the present disclosure are directed to a method of preventing an increase of volume of a tumor, wherein the tumor is present in a body of a living subject and includes a plurality of cancer cells. The method includes the steps of: (1) applying an alternating electric field to a target region of the subject for a period of time; and (2) administering at least one composition to the subject, wherein the at least one composition comprises at least one killer cell (such as, but not limited to, at least one natural killer (NK) cell or at least one cytokine-induced killer (CIK) cell).

Certain additional non-limiting embodiments of the present disclosure are directed to a method, comprising the steps of: (1) applying an alternating electric field to a target region of the subject for a period of time; and (2) administering at least one composition to the subject, wherein the at least one composition comprises at least one killer cell (such as, but not limited to, at least one natural killer (NK) cell or at least one cytokine-induced killer (CIK) cell).

Certain additional non-limiting embodiments of the present disclosure are directed to a composition comprising at least one natural killer (NK) cell, for use in a method of treating cancer in a subject, the method comprising the steps of: (1) applying an alternating electric field to a target region of the subject for a period of time; and (2) administering the composition to the subject, wherein the composition comprises at least one natural killer (NK) cell.

Certain additional non-limiting embodiments of the present disclosure are directed to a kit for reducing viability of cancer cells, the kit comprising: at least one composition comprising at least one natural killer (NK) cell; and a field generating device configured to apply an alternating electric field to the cancer cells for a period of time.

Certain additional non-limiting embodiments of the present disclosure are directed to a kit for reducing viability of cancer cells, the kit comprising: components configured for isolating and culturing at least one natural killer cell, or a precursor thereof, from a subject; and a field generating device configured to apply an alternating electric field to the cancer cells for a period of time.

In certain particular (but non-limiting) embodiments, administration of the alternating electric field in any of the methods described herein above or otherwise herein increases the efficacy and/or cytotoxicity of the killer cells against cancer cells in the subject when compared to the administration of killer cells to the subject in the absence of alternating electric field application.

Steps (1) and (2) of any of the methods of the present disclosure may be performed concomitantly or serially, and in particular, substantially simultaneously or wholly or partially sequentially. When the steps are performed wholly or partially sequentially, the at least one composition comprising at least one killer cell may be administered before or after application of the alternating electric field has begun.

The methods of the present disclosure may be utilized to treat any types of cancer cells/cancers/tumors that respond to treatment with alternating electric fields (e.g., TTFields) and/or killer cells. Non-limiting examples of cancer cells/cancers/tumors that can be treated in accordance with the present disclosure include hepatocellular carcinoma/carcinoma cells, glioblastoma/glioblastoma cells, pleural mesothelioma/mesothelioma cells, differentiated thyroid cancer/cancer cells, advanced renal cell carcinoma/carcinoma cells, ovarian cancer/cancer cells, pancreatic cancer/cancer cells, lung cancer/cancer cells, breast cancer/cancer cells, and the like, as well as any combination thereof.

Any type of conductive or non-conductive electrode(s) and/or transducer array(s) that can be utilized for generating an alternating electric field that are known in the art or otherwise contemplated herein may be utilized for generation of the alternating electric field in accordance with the methods of the present disclosure. Non-limiting examples of electrodes and transducer arrays that can be utilized for generating an alternating electric field in accordance with the present disclosure include those that function as part of a TTFields system as described, for example but not by way of limitation, in U.S. Pat. Nos. 7,016,725; 7,089,054; 7,333,852; 7,565,205; 8,244,345; 8,715,203; 8,764,675; 10,188,851; and 10,441,776; and in US Patent Application Nos. US 2018/0160933; US 2019/0117956; US 2019/0307781; and US 2019/0308016.

The alternating electric field may be generated at any frequency in accordance with the present disclosure. For example (but not byway of limitation), the alternating electric field may have a frequency of about 50 kHz, about 75 kHz, about 100 kHz, about 125 kHz, about 150 kHz, about 175 kHz, about 200 kHz, about 225 kHz, about 250 kHz, about 275 kHz, about 300 kHz, about 325 kHz, about 350 kHz, about 375 kHz, about 400 kHz, about 425 kHz, about 450 kHz, about 475 kHz, about 500 kHz, about 550 kHz, about 600 kHz, about 650 kHz, about 700 kHz, about 750 kHz, about 800 kHz, about 850 kHz, about 900 kHz, about 950 kHz, about 1 MHz, about 2 MHz, about 3 MHz, about 4 MHz, about 5 MHz, about 6 MHz, about 7 MHz, about 8 MHz, about 9 MHz, about 10 MHz, and the like, as well as a range formed from any of the above values (e.g., a range of from about 50 kHz to about 10 MHz, a range of from about 50 kHz to about 1 MHz, a range of from about 50 kHz to about 500 kHz, a range of from about 100 kHz to about 500 kHz, a range of from about 150 kHz to about 300 kHz, etc.), and a range that combines two integers that fall between two of the above-referenced values (e.g., a range of from about 122 kHz to about 313 kHz, a range of from about 78 kHz to about 298 kHz, etc.).

In certain particular (but non-limiting) embodiments, the alternating electric field may be imposed at two or more different frequencies. When two or more frequencies are present, each frequency is selected from any of the above-referenced values, or a range formed from any of the above-referenced values, or a range that combines two integers that fall between two of the above-referenced values.

In certain particular (but non-limiting) embodiments, the following frequencies may be utilized for specific cancers: GBM, about 200 kHz; NSCLC, about 150 kHz; breast cancer, about 200 kHz; pancreatic cancer, about 150 kHz; brain metastases from NSCLC, about 150 kHz; hepatic cancer, about 150 kHz; and the like.

The alternating electric field may have any field strength in the target region/subject/cancer cells, so long as the alternating electric field is capable of functioning in accordance with the present disclosure. For example (but not by way of limitation), the alternating electric field may have a field strength in the target region/subject/cancer cells of at least about 1 V/cm, about 1.5 V/cm, about 2 V/cm, about 2.5 V/cm, about 3 V/cm, about 3.5 V/cm, about 4 V/cm, about 4.5 V/cm, about 5 V/cm, about 5.5 V/cm, about 6 V/cm, about 6.5 V/cm, about 7 V/cm, about 7.5 V/cm, about 8 V/cm, about 9 V/cm, about 9.5 V/cm, about 10 V/cm, about 10.5 V/cm, about 11 V/cm, about 11.5 V/cm, about 12 V/cm, about 12.5 V/cm, about 13 V/cm, about 13.5 V/cm, about 14 V/cm, about 14.5 V/cm, about 15 V/cm, about 15.5 V/cm, about 16 V/cm, about 16.5 V/cm, about 17 V/cm, about 17.5 V/cm, about 18 V/cm, about 18.5 V/cm, about 19 V/cm, about 19.5 V/cm, about 20 V/cm, and the like, as well as a range formed from any of the above values (e.g., a range of from about 1 V/cm to about 20 V/cm, a range of from about 1 V/cm to about 10 V/cm, a range of from about 1 V/cm to about 4 V/cm, etc.), and a range that combines two integers that fall between two of the above-referenced values (e.g., a range of from about 1.1 V/cm to about 18.6 V/cm, a range of from about 1.2 V/cm to about 9.8 V/cm, a range of from about 1.3 V/cm to about 4.7 V/cm, etc.). Generally, it is desired to utilize the highest field strength possible without causing overheating, with field intensity typically being capped by temperature measurements.

In some instances, the electric field in at least a portion of the target region/subject/cancer cells is induced by an applied voltage that is determined by computer simulation of the target region/subject/cancer cells. In some instances, the electric field in at least a portion of the target region/subject/cancer cells is induced by an applied voltage of at least 50 V RMS (root mean squared) or at least 50 V p2p (peak-to-peak), and optionally, the applied voltage is at least 100 V RMS or at least 100 V p2p. In some embodiments, an applied voltage of at least 50 V induces an electric field with a field strength of at least 1 V/cm (e.g., at least 5 V/cm) in at least a portion of the target region/subject/cancer cells.

The alternating electric field may be applied in a single direction between a pair of arrays or may be alternating in two or more directions/channels between two or more pairs of arrays (e.g., front-back and left-right). For example, certain TTFields devices (such as, but not limited to, the OPTUNE® system (Novocure Limited, St. Helier, Jersey)) operate in two directions in order to increase the chances that a dividing cell will be aligned with the electric field such that the electric field can have the desired anti-mitotic effect. However, it will be understood that the scope of the present disclosure also includes the application of the alternating electric field in a single direction. The term “alternating electric field” as used herein will be understood to include application in a single direction/channel as well as in two or more directions/channels; in addition, the term “alternating electric field” as used herein will be understood to include both application of a single alternating electric field as well as application of a plurality of alternating electric fields in succession for a duration of time.

The alternating electric field may be applied for any continuous or cumulative period of time sufficient to achieve a reduction in viability of cancer cells and/or a reduction in tumor volume (and/or a prevention of increase in tumor volume). The period of time that the alternating electric field is applied includes both a continuous period of time as well as a cumulative period of time. That is, the period of time that the alternating electric field is applied includes a single session (i.e., continuous application) as well as multiple sessions with minor breaks in between sessions (i.e., consecutive application for a cumulative period). For example, a subject is allowed to take breaks during treatment with an alternating electric field device and is only expected to have the device positioned on the body and operational for at least about 60%, at least about 70%, or at least about 80% of the total treatment period (e.g., over a course of one day, one week, two weeks, one month, two months, three months, four months, five months, etc.).

For example, but not by way of limitation, the alternating electric field may be applied for a continuous or cumulative period of time of at least about 1 hour, about 2 hours, about 3 hours, about 4 hours, about 5 hours, about 6 hours, about 7 hours, about 8 hours, about 9 hours, about 10 hours, about 11 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, about 5 days, about 6 days, about 7 days, about 8 days, about 9 days, about 10 days, about 11 days, about 12 days, about 13 days, about 14 days, about 21 days, about 1 month, about 2 months, about 3 months, about 4 months, about 5 months, about 6 months, and the like, as well as a range formed from any of the above values (e.g., a range of from about 1 hour to about 6 months, a range of from about 24 hours to about 72 hours, etc.), and a range that combines two integers that fall between two of the above-referenced values (e.g., a range of from about 14 hours to about 68 hours, etc.).

In a particular (but non-limiting) embodiment, the period of time that the alternating electric field is applied is at least about 24 hours, with the device positioned on the body and operational for at least about 80% of that period.

In a particular (but non-limiting) embodiment, the alternating electric field is applied to the cancer cells/target region of the subject for a period of time sufficient to reduce MHC Class I expression in the cancer cells compared to control cancer cells that have not been exposed to alternating electric fields.

Any killer cells that are known in the art or are otherwise contemplated herein may be utilized in accordance with the present disclosure, so long as the killer cells are capable of functioning as described herein. Non-limiting examples of killer cells that may be utilized in accordance with the present disclosure include natural killer (NK) cells and cytokine-induced killer (CIK) cells. The killer cells may be autologous (and thus obtained from the subject to be treated), or the killer cells may be allogeneic (and thus obtained from another subject). The autologous or allogeneic killer cells could be cultured directly from a subject and then implanted/injected into the cancer patient. Alternatively (and/or in addition thereto), pluripotent stem cells may be obtained from a subject and differentiated ex vivo into killer cells that are subsequently implanted/injected into the cancer patient.

In certain particular (but non-limiting embodiments), the killer cells are derived from an interleukin-2 (IL-2) dependent cell line. For example (but not by way of limitation), NK-92© cells ImmunityBio, Inc., Culver City, CA) are commercially available and may be utilized in accordance with the present disclosure

In certain particular (but non-limiting) embodiments, the killer cells are engineered. For example, but not by way of limitation, the killer cells may include chimeric antigen receptor-engineered NK (CAR-NK) cells.

Various other types of killer cells that may be utilized in accordance with the present disclosure are known in the art and commercially available from multiple sources; therefore, no further description thereof is deemed necessary.

The composition comprising at least one killer cell may be provided with any formulation known in the art or otherwise contemplated herein. In certain particular (but non-limiting) embodiments, the composition comprising at least one killer cell contains one or more pharmaceutically acceptable carriers (and as such, the composition may also be referred to as a “pharmaceutical composition”). Non-limiting examples of suitable pharmaceutically acceptable carriers include water; saline; dextrose solutions; fructose or mannitol; calcium carbonate; cellulose; ethanol; oils of animal, vegetative, or synthetic origin; carbohydrates, such as glucose, sucrose, or dextrans; antioxidants, such as ascorbic acid or glutathione; chelating agents; low molecular weight proteins; detergents; liposomal carriers; buffered solutions, such as sodium chloride, saline, phosphate-buffered saline, and/or other substances which are physiologically acceptable and/or safe for use; diluents; excipients such as polyethylene glycol (PEG); or any combination thereof. Suitable pharmaceutically acceptable carriers for pharmaceutical formulations are described, for example, in Remington: The Science and Practice of Pharmacy, 23rd ed. (2020).

In certain particular (but non-limiting) embodiments, the composition comprising at least one killer cell may further contain one or more additional active agents. Various active agents that can be utilized concurrently with alternating electric fields or killer cells are known in the art, and certain combination therapies are approved by the FDA or currently in clinical trials testing. Non-limiting examples of therapeutic agents that can be utilized in accordance with the present disclosure concurrently with killer cells include anti-PD-1 therapeutics such as (but not limited to) Pembrolizumab (KEYTRUDA®, Merck & Co., Inc., Rahway, NJ), Tislelizumab, Nivolumab, and Cemiplimab; anti-PD-L1 therapeutics such as atezolizumab, avelumab, and durvalumab; chemotherapeutic agents, such as (but not limited to) lenvatinib; Paclitaxel, Docetaxel, Ifosamide, Etoposide (VEPESID®, Bristol-Myers Squibb Co, New York, NY), Gemcitabine, Lomustine, nab Paclitaxel, temozolomide, and Carboplatin; TKI inhibitors, such as (but not limited to) Everolimus; mTOR inhibitors; Akt inhibitors; PI3K inhibitors; PARP inhibitors; FGF inhibitors; anti-LAB3 agents; anti-CTLA-4 therapeutics; aromatase inhibitors, such as (but not limited to) Letrozole; biologics such as monoclonal antibodies (such as, but not limited to, Denosumab and pembrolizumab); and the like, as well as any combinations thereof.

In addition, any of the compositions of the present disclosure may contain other agents that allow for administration of the compositions via a particular administration route. For example, but not by way of limitation, the compositions may be formulated for administration by oral, topical, transdermal, parenteral, subcutaneous, intranasal, mucosal, intramuscular, intraperitoneal, intravitreal, and/or intravenous routes. Based on the route of administration, the compositions may also contain one or more additional components in addition to the active agent (e.g., killer cell(s) and/or additional therapeutic agent(s)). Examples of additional secondary compounds that may be present include, but are not limited to, fillers, salts, buffers, preservatives, stabilizers, solubilizers, wetting agents, emulsifying agents, dispersing agents, and other materials well known in the art.

In a particular (but non-limiting) embodiment, the at least one composition comprising the at least one killer cell is administered via injection or implantation into the subject. For example (but not by way of limitation), in some instances, it may be desired that the killer cell(s) be administered on a local/regional level to ensure targeting of the killer cells to a specific location in the body of the subject and inhibit non-specific interactions in other parts of the body; in other instances, a more systemic administration may be desired.

The at least one composition comprising at least one killer cell may be administered before or after application of the alternating electric field has begun. In certain particular (but non-limiting) embodiments, the at least one composition comprising at least one killer cell may be administered after the application of the alternating electric field has begun. In particular (but not by way of limitation), the at least one composition comprising at least one killer cell may be administered during application of the alternating electric field (e.g., before the period of time that the alternating electric field is applied has elapsed) and/or after application of the alternating electric field has elapsed.

For example (but not by way of limitation), the at least one composition comprising the at least one killer cell may be administered after application of the alternating electric field has commenced by a period of at least about 3 hours, about 6 hours, about 9 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, about 5 days, about 6 days, about 7 days, and the like, as well as a range formed from any of the above values (e.g., a range of from about 24 hours to about 96 hours, etc.), and a range that combines two integers that fall between two of the above-referenced values (e.g., a range of from about 14 hours to about 94 hours, etc.). In a particular (but non-limiting) embodiment, the at least one composition comprising the at least one killer cell is administered at least about 24 hours after application of the alternating electric field has begun.

In other non-limiting examples, the at least one composition comprising at least one killer cell may be administered after the period of time that the alternating electric field is applied has elapsed, wherein the at least one composition comprising at least one killer cell is administered within about 3 hours, about 6 hours, about 9 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, about 5 days, about 6 days, about 7 days, and the like, of when the period of time elapsed.

In a particular (but non-limiting) embodiment, the at least one composition comprising at least one killer cell is administered within about 96 hours of when the period of time elapsed.

The composition comprising at least one killer cell may be administered to the cancer cells/subject at any concentration that provides a therapeutically effective concentration of killer cells. In certain non-limiting embodiments, the application of the alternating electric field reduces the amount of killer cells required to be therapeutically effective when compared to a normal therapeutically effective amount administered in the absence of an alternating electric field. For example, but not by way of limitation, the therapeutically effective concentration of killer cells may be reduced by at least about 25%, about 30%, about 35%, about 40%, about 45%, about 50%, about 55%, about 60%, about 65%, about 70%, about 75% or more with respect to a dosage of killer cells known to be therapeutically effective in the absence of application of an alternating electric field. In a particular (but non-limiting) embodiment, the therapeutically effective concentration of killer cells is reduced by at least about 50% when compared to a dosage of killer cells known to be therapeutically effective in the absence of an alternating electric field.

The killer cell-containing composition(s) may be administered to the subject at any concentration that is capable of inducing an inflammatory response to the tumor or cancer cells. For example, but not by way of limitation, the killer cells may be administered at about 10 cells/kg body weight, about 100 cells/kg body weight, about 1000 cells/kg body weight, about 10⁴ cells/kg body weight, about 10⁵ cells/kg body weight, about 10⁶ cells/kg body weight, about 10⁷ cells/kg body weight, about 10⁸ cells/kg body weight, about 10⁹ cells/kg body weight, about 10¹⁰ cells/kg body weight, about 10¹¹ cells/kg body weight, about 10¹² cells/kg body weight, about 10¹³ cells/kg body weight, about 10¹⁴ cells/kg body weight, about 10¹⁵ cells/kg body weight, or higher, as well as a range formed from any of the above values (e.g., a range of from about 10⁴ to about 10⁹ cells/kg body weight, etc.).

In certain particular (but non-limiting) embodiments, the method includes one or more additional steps. For example (but not by way of limitation), the method may further include the step of (3) discontinuing the application of the alternating electric field (such as, but not limited to, to allow the cells/tissue to recover). In addition, any of steps (1) and/or (2) may be repeated one or more times.

In certain particular (but non-limiting) embodiments, the killer cell-containing composition may be administered by any dosage regimen known in the art. For example, but not by way of limitation, an killer cell-containing composition may be administered in a single dosage or multiple dosages over a defined treatment period. For example (but not by way of limitation), a therapeutically effective concentration of the killer cell-containing composition may be administered about once every 4 hours, about once every 8 hours, about once every 12 hours, about once every day, about once every other day, about once every three days, about once a week, about twice a week, about three times a week, about once every two weeks, about once every three weeks, about once a month, and the like, as well as a range formed from any of the above values (a range of about once every 4 to 8 hours, a range of from about once a week to about once a month, etc.).

In certain particular (but non-limiting) embodiments, the method involves concurrent therapy with two or more compositions. As such, the method may include an additional step of (4) administering at least a second composition to the cancer cells/subject. In a particular (but non-limiting) embodiment, the at least second composition may contain one or more of any of the active substances disclosed or otherwise contemplated herein for use with the killer cell-containing composition.

Various substances and therapies utilized concurrently with killer cells are known in the art, and certain combination therapies are approved by the FDA or currently in clinical trials testing. Non-limiting examples of therapeutic agents that can be utilized in accordance with the present disclosure in concurrent (sequential) therapy with killer cells include anti-PD-1 therapeutics such as (but not limited to) Pembrolizumab (KEYTRUDA®, Merck & Co., Inc., Rahway, NJ), Tislelizumab, Nivolumab, and Cemiplimab; anti-PD-L1 therapeutics such as atezolizumab, avelumab, and durvalumab; chemotherapeutic agents, such as (but not limited to) lenvatinib; Paclitaxel, Docetaxel, Ifosamide, Etoposide (VEPESID®, Bristol-Myers Squibb Co, New York, NY), Gemcitabine, Lomustine, nab Paclitaxel, temozolomide, and Carboplatin; TKI inhibitors, such as (but not limited to) Everolimus; mTOR inhibitors; Akt inhibitors; PI3K inhibitors; PARP inhibitors; FGF inhibitors; anti-LAB3 agents; anti-CTLA-4 therapeutics; aromatase inhibitors, such as (but not limited to) Letrozole; biologics such as monoclonal antibodies (such as, but not limited to, Denosumab and pembrolizumab); and the like, as well as any combinations thereof.

When present, step (4) may be performed substantially simultaneously or wholly or partially sequentially with the administration of the first composition in step (2), whereby the two separate compositions are administered simultaneously or wholly or partially sequentially. In addition, the two compositions administered in steps (2) and (4) may be administered via the same route (e.g., both administered intravenously), or the two compositions may be administered by different routes (e.g., one composition orally administered and another composition intravenously administered).

When present, the optional additional administration step (4) may be performed before or after the application of the alternating electric field has begun, during application of the alternating electric field, and/or after application of the alternating electric field has elapsed, in the same manner(s) and time frame(s) as described above for the first composition.

That is, for example (but not by way of limitation), the second composition may be administered after application of the alternating electric field has commenced by a period of at least about 3 hours, about 6 hours, about 9 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, about 5 days, about 6 days, about 7 days, and the like, as well as a range formed from any of the above values (e.g., a range of from about 24 hours to about 96 hours, etc.), and a range that combines two integers that fall between two of the above-referenced values (e.g., a range of from about 14 hours to about 94 hours, etc.). In a particular (but non-limiting) embodiment, the second composition is administered at least about 24 hours after application of the alternating electric field has begun.

In other non-limiting examples, the second composition may be administered after the period of time that the alternating electric field is applied has elapsed, wherein the second composition is administered within about 3 hours, about 6 hours, about 9 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, about 5 days, about 6 days, about 7 days, and the like, of when the period of time elapsed. In a particular (but non-limiting) embodiment, the second composition is administered within about 96 hours of when the period of time elapsed.

In addition, for example (but not by way of limitation), the second composition may be administered after administration of the first substance by a period of at least about 1 minute, about 5 minutes, about 10 minutes, about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, about 5 days, about 6 days, about 7 days, and the like, as well as a range formed from any of the above values (e.g., a range of from about 24 hours to about 96 hours, etc.), and a range that combines two integers that fall between two of the above-referenced values (e.g., a range of from about 14 hours to about 94 hours, etc.). In a particular (but non-limiting) embodiment, the second composition is administered at least about 12 hours after administration of the first substance.

In certain particular (but non-limiting) embodiments, the method may further comprise the step of (5) administering at least one additional therapy to the cells/subject. Any therapies known in the art or otherwise contemplated herein for use with alternating electric fields (e.g., TTFields) and/or killer cell therapy may be utilized in accordance with the methods of the present disclosure. Non-limiting examples of additional therapies that may be utilized include radiation therapy, photodynamic therapy, transarterial chemoembolization (TACE), or combinations thereof.

Any of steps (1) and (2) and optional steps (3), (4), and (5) may be repeated one or more times. Each of the steps can be repeated as many times as necessary. When step (1) is repeated, the transducer arrays may be placed in slightly different positions on the subject than their original placement; relocation of the arrays in this manner may further aid in treatment of the tumor/cancer. In addition, step (2) and optional steps (4) and (5) (when present) of administering compositions/additional therapies may be repeated various times and at various intervals to follow any known and/or generally accepted dosage/treatment regimen for the composition(s)/therapy(ies).

The use of ordinal references to the optional steps is for purpose of example only; the methods of the present disclosure may include one or more of the optional steps (3), (4), and (5), either alone or in combination with one another. That is, the methods of the present disclosure include performing step (3) in the absence of steps (4) or (5), performing step (4) in the absence of steps (3) or (5), and performing step (5) in the absence of steps (3) and (4). In other words, the scope of the methods disclosed herein includes performing steps (1)-(2) (as well as repeating each step as many times as necessary), performing steps (1)-(3) (as well as repeating one or more of steps (1)-(3) as many times as necessary), performing steps (1)-(2) and (4) (as well as repeating one or more of steps (1)-(2) and (4) as many times as necessary), performing steps (1)-(2) and (5) (as well as repeating one or more of steps (1)-(2) and (5) as many times as necessary), performing steps (1)-(4) (as well as repeating one or more of steps (1)-(4) as many times as necessary), performing steps (1)-(3) and (5) (as well as repeating one or more of steps (1)-(3) and (5) as many times as necessary), performing steps (1)-(2) and (4)-(5) (as well as repeating one or more of steps (1)-(2) and (4)-(5) as many times as necessary), and performing all of steps (1)-(5) (as well as repeating one or more of steps (1)-(5) as many times as necessary).

While the use of concurrent therapy with two substances is explicitly described above, it will be understood that the scope of the present disclosure further includes concurrent therapy with three or more compositions. As such, the method can include one or more additional steps of administering an additional composition to the subject (similar to steps (1) and (4)). Any additional substances administered in the method may be selected from any of the substances disclosed or otherwise contemplated herein for use concurrently with at least one killer cell (as disclosed herein above with respect to optional step (4)); in addition, administration of any additional substances can be performed substantially simultaneously or wholly or partially sequentially with the administration of the first and/or second compositions/substances and in the same manner(s) and time frame(s) as described above for the first and second compositions/substances.

Certain non-limiting embodiments of the present disclosure are related to kits that include any of the components of the alternating electric field (e.g., TTFields) generating systems disclosed or otherwise contemplated herein (such as, but not limited to, one or more transducer arrays and/or one or more hydrogel compositions, as disclosed in U.S. Pat. Nos. 7,016,725; 7,089,054; 7,333,852; 7,565,205; 8,244,345; 8,715,203; 8,764,675; 10,188,851; and 10,441,776; and in US Patent Application Nos. US 2018/0160933; US 2019/0117956; US 2019/0307781; and US 2019/0308016) in combination with at least one of any of the compositions comprising at least one killer cell disclosed or otherwise contemplated herein, and/or components for isolating and/or culturing at least one killer cell or a precursor thereof from a patient. The kits may optionally further include one or more of any of the optional compositions disclosed or otherwise contemplated herein (such as, but not limited to, one or more optional compositions containing at least one additional active agent and/or one or more optional compositions for culturing/inducing differentiation of the at least one killer cells or precursor thereof). The kits may optionally further include one or more devices (or one or more components of devices) utilized in one or more additional therapy steps.

In a particular (but non-limiting) embodiment, the kit may further include instructions for performing any of the methods disclosed or otherwise contemplated herein. For example (but not by way of limitation), the kit may include instructions for applying one or more components of the alternating electric field (e.g., TTFields) generating device to the skin of the patient, instructions for applying the alternating electric field to the patient, instructions for isolating the killer cell(s) or precursor(s) thereof from a subject, instructions for culturing and/or differentiating the killer cell(s) or precursor(s) thereof ex vivo, instructions for formulating the composition comprising the at least one killer cell, instructions for when and how to administer the composition comprising the at least one killer cell and optionally how to administer one or more optional additional compositions, and/or instructions for when to activate and turn off the alternating electric field in relation to the administration of the composition comprising the at least one killer cell and/or administration of one or more optional compositions and/or therapy steps.

In addition to the components described in detail herein above, the kits may further contain other component(s)/reagent(s) for performing any of the particular methods described or otherwise contemplated herein. For example (but not byway of limitation), the kits may additionally include: (i) components for preparing the skin prior to disposal of the hydrogel compositions and/or transducer arrays thereon (e.g., a razor, a cleansing composition or wipe/towel, etc.); (ii) components for removal of the gel/transducer array(s); (iii) components for cleansing of the skin after removal of the gel/transducer array(s); and/or (iv) other components utilized with the system (e.g., conductive material, nonconductive material, a soothing gel or cream, a bandage, etc.). The nature of these additional component(s)/reagent(s) will depend upon the particular treatment format, and identification thereof is well within the skill of one of ordinary skill in the art; therefore, no further description thereof is deemed necessary. Also, the components/reagents present in the kits may each be in separate containers/compartments, or various components/reagents can be combined in one or more containers/compartments, depending on the sterility, cross-reactivity, and stability of the components/reagents.

The kit may be disposed in any packaging that allows the components present therein to function in accordance with the present disclosure. In certain non-limiting embodiments, the kit further comprises a sealed packaging in which the components are disposed. In certain particular (but non-limiting) embodiments, the sealed packaging is substantially impermeable to air and/or substantially impermeable to light.

In addition, the kit can further include a set of written instructions explaining how to use one or more components of the kit. A kit of this nature can be used in any of the methods described or otherwise contemplated herein.

In certain non-limiting embodiments, the kit has a shelf life of at least about six months, such as (but not limited to), at least about nine months, or at least about 12 months.

Certain non-limiting embodiments of the present disclosure are related to systems that include any of the components of the alternating electric field generating systems disclosed or otherwise contemplated herein (such as, but not limited to, one or more transducer arrays and/or one or more hydrogel compositions, as disclosed in U.S. Pat. Nos. 7,016,725; 7,089,054; 7,333,852; 7,565,205; 8,244,345; 8,715,203; 8,764,675; 10,188,851; and 10,441,776; and in US Patent Application Nos. US 2018/0160933; US 2019/0117956; US 2019/0307781; and US 2019/0308016) in combination with at least one of any of the compositions comprising at least one killer cell disclosed or otherwise contemplated herein. The systems may optionally further include one or more of any of the optional compositions disclosed or otherwise contemplated herein. The systems may optionally further include one or more devices (or one or more components of devices) utilized in one or more additional therapy steps.

### EXAMPLES

Examples are provided herein below. However, the present disclosure is to be understood to not be limited in its application to the specific experimentation, results, and laboratory procedures disclosed herein after. Rather, the Examples are simply provided as one of various embodiments and is meant to be exemplary, not exhaustive.

### Example 1

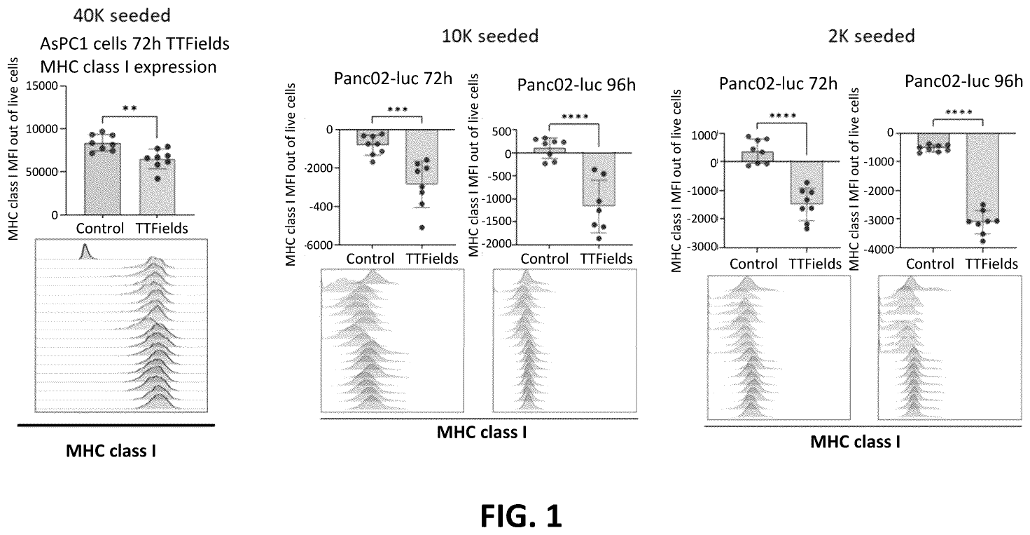

In this Example, the effects of TTFields on MHC class I expression in cancer cell lines was examined. In contrast to the prior art of Silginer et al. (2018) referenced in the Background section above, which found that MHC Class I and Class 11 expression remained unaltered following exposure to TTFields, this Example found that MHC class I expression was actually reduced in human and mouse pancreatic cancer cell lines. As shown in FIG. 1, MHC class I expression was reduced in the 1A-AsPc1 human pancreatic cancer cell line following a 72-hour exposure to TTFields (left panel), and MHC class I expression was reduced in the 1B-Panc02 mouse pancreatic cancer cell line following 72- or 96-hour exposures to TTFields (middle and right panels).

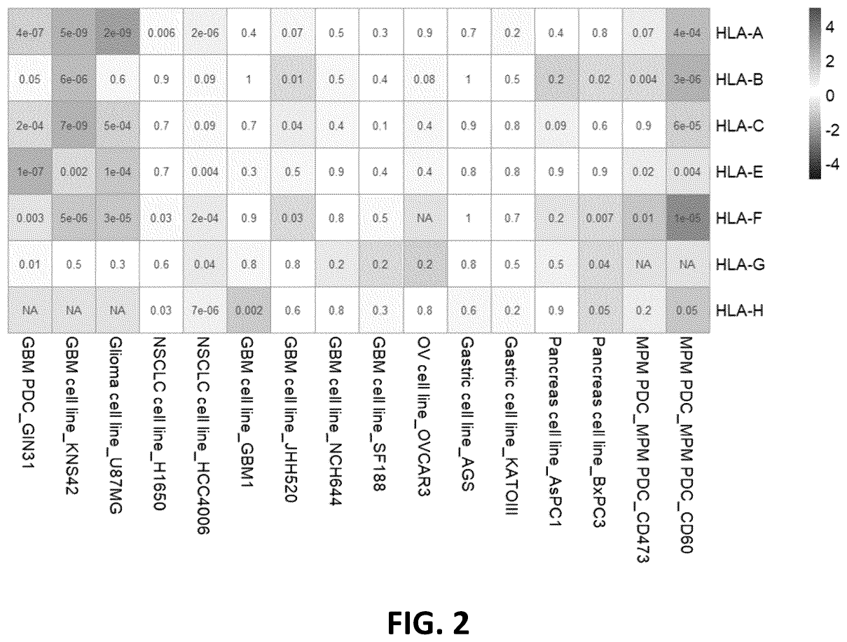

Furthermore, a transcriptomics analysis was conducted to identify class I HLA genes that exhibit differences in expression following TTFields treatment of various cancer cell lines compared with control. As shown in FIG. 2, there is a statistically significant reduction in various class I HLAs tested (including HLA-A, HLA-B, HLA-C, HLA-E, HLA-F, HLA-G, and HLA-H) following TTFields in glioblastoma (GBM) cell lines, malignant pleural mesothelioma cell line (MPM), and pancreatic cancer cell lines AsPC1 and BxPC3. Therefore, this data demonstrates that TTFields decrease MHC class I expression in various cancers.

While not wishing to be bound by any theory, it has previously been shown that TTFields induce ER stress (Silginer et al. (2017) Cell Death Dis, 8:e2753). In addition, it has also been shown previously that ER stress downregulates class I HLA (Ulianich et al. (2011) Biochemica et. Biophysica Acta, 1812:431-438). Therefore, the induction of ER stress by TTFields exposure could be a possible mechanism for downregulation of class I HLA following TTFields exposure.

### Example 2

This Example is directed to the use of TTFields as an immunotherapy to activate adoptive-transferred NK cells to increase the efficacy of NK cell-based cancer therapies. The concurrent therapy of TTFields with adoptive-transferred NK cells provides an improved and synergistic effect over either treatment alone.

Human subjects are treated with TTFields by application of an OPTUNE® device (Novocure Limited, St. Helier, Jersey) to the skin of the subject, with placement of the pair of arrays left and right (LR) of the tumor and/or anterior and posterior (AP) to the tumor. Each subject is then chronically treated with TTFields at 150-200 kHz; the device is worn at least about 80% of the time, with minor breaks in between sessions and slight adjustments to the placements of the arrays, to allow the cells and skin to recover.

A composition containing NK cells is prepared. In one instance, autologous NK cells are obtained from the subject prior to TTFields exposure. Alternatively, NK cells are obtained from an allogeneic source. In yet another example, pluripotent stem cells are obtained from an autologous of allogeneic source and differentiated to NK cells ex vivo. The autologous or allogeneic NK cells are then cultured prior to implantation/injection for adoptive cell transfer therapy.

Two weeks after application of the TTFields has commenced, approximately 1-10 billion NK cells are implanted or injected in the human subjects about 1-3 times a week for 6-8 weeks.

Following the concurrent therapy treatment, the effects of said concurrent therapy on the tumor(s) is evaluated.

### Example 3

This Example studies the role of TTFields in potentiating the killing activity of CIK cells in vitro against patient-derived GBM cancer stem cells (GSCs). This Example provides proof of principle that TTFields can potentiate “HLA-independent immunity” against cancer cells, and also strengthens the basis for novel clinical studies that revisit the potential of CIK therapy in combination with TTFields to treat GBM.

Cytokine-induced killer (CIK) cell therapy is a type of adoptive immunotherapy that uses a heterogeneous population of immune cells generated ex vivo from patient peripheral blood mononuclear cells (PBMCs) in the presence of IL-2, anti-CD3 antibody(ies) (such as, but not limited to, OKT3), and IFN-gamma (Introna (2017) Journal of Autoimmunity, 85:32-44). The CIK cells include a CD3⁺CD56⁺CD8⁺ population, which are phenotypically cytotoxic T-terminally differentiated effector memory (EMRA) lymphocytes (Franceschetti et al. (2009) Experimental Hematology, 37:616-628.e2) with antitumor activity. This population expresses different NK markers, including NKG2D, and is able to recognize and kill tumor cells in a TCR-independent, NK-like manner (Sangiolo et al. (2008) International Immunology, 20:841-848). According to a recent report from the International Registry on CIK Cells (IRCC), CIK cells have been tested in the setting of 106 clinical trials in over 30 distinct tumors, with a significant improvement in progression-free survival (PFS) and overall survival (OS) in 27 trials in the autologous setting, with mild adverse effects. CIK cells have also been evaluated in patients with GBM, the most common malignant primary brain tumor (Kong et al. (2017) Oncotarget, 8:7003-7013). In this phase III trial, CIK cells were associated with improvement in PFS but not OS. This trial is one of several immunotherapy trials with poor clinical outcomes in GBM, including immune checkpoint inhibitors, vaccinations, and different adoptive T cell therapies (Weenink et al. (2020) Cancers (Basel), 12:E751).

TTFields have been shown to extend survival for patients with both newly diagnosed and recurrent GBM, leading to clinical approval of this therapy by the FDA (Rominiyi et al. (2020) Br J Cancer, 124(4):697-709). While not wishing to be bound by any theory, the main biological mechanism attributed to TTFields in cancer therapy is its ability to interfere with the formation of the mitotic spindle during mitotic apparatus (Kirson et al. (2004) Cancer Res, 1; 64(9):3288-95). However, emerging evidence indicates that TTFields are also able to activate the immune system against tumor cells (Voloshin et al. (2020) Cancer Immunol Immunother, 69(7):1191-1204). Chen et al. (J Clin Invest, 132(8):e149258 (2022)) have shown mechanistically, in a syngeneic murine GBM model, that TTFields, through the genotoxic stress activation of cGAS/STING- and AIM2 inflammasome in GBM cells, are able to stimulate the production of proinflammatory and interferon type 1 cytokines. This proinflammatory shift is able to produce a robust activation of adaptive immunity, showing an increase in T cell clonal expansion with memory phenotype, and a reduction in exhausted T cells. Despite the single-cell RNA sequencing of peripheral blood mononuclear cells obtained from GBM patients treated with TTFields displayed clues of activation also in NK cells, the role of “HLA-independent immunity” was not further explored in Chen et al.

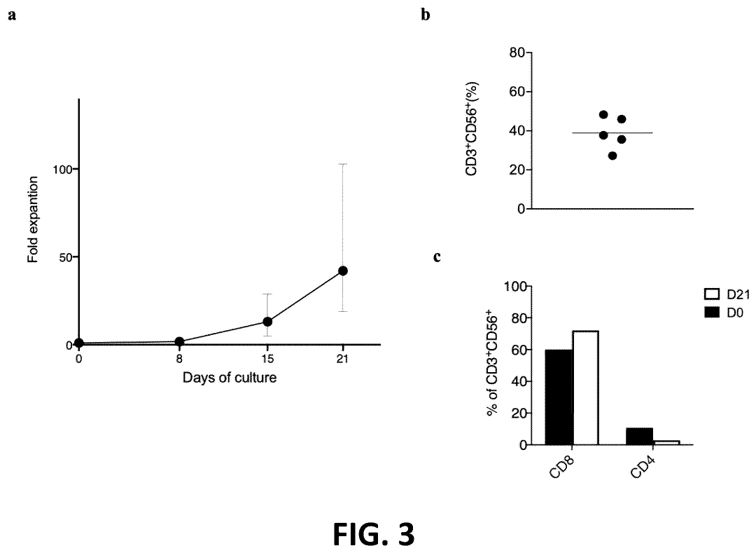

In this Example, 20 batches of CIK were successfully expanded from PBMCs. The PBMCs were obtained both from healthy donors (HD) and from patients with GBM before chemo-radiotherapy treatment enrolled at San Raffaele Hospital, following the protocol described in the Methods section below. FIG. 3 summarizes the growth curve of the 5 batches obtained from 5 HDs, which displayed a median expansion fold of 42.03 (IQR:31.15-74.13). At day 21, CD3⁺CD56⁺ cells represented 38.96% of the cells, and this population was mostly CD8⁺CD4⁻. CD3⁺CD56⁻, which represented 48.83% of the cells, resulted mostly as CD8⁺CD4⁻ cells (mean=61.43%) but presented a more significant proportion of CD4⁺CD8⁻ cells (mean=28.94%).

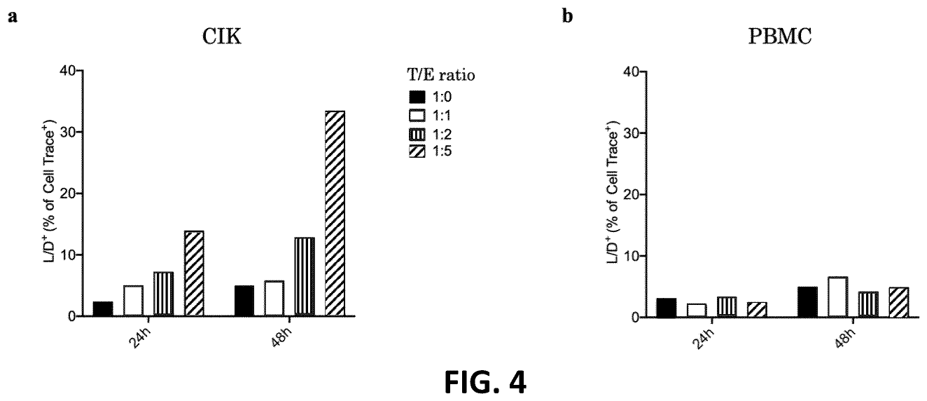

CIK and GSC co-culture: In order to assess the cytotoxic activity of CIK cells, their ability to induce apoptosis in patient-derived GSC was tested. To do this, GSC were labeled with violet CellTrace Cell Proliferation Kit and then co-cultured with CIK cells with different target-to-effector ratios (T/E ratio). As shown in FIG. 4, a dose-dependent cytotoxic effect induced by CIK was observed as compared with fresh PBMC.

### Research Design and Methods

### CIK Cells

Blood samples were collected in EDTA from healthy volunteers. PBMCs were separated from whole blood through density centrifugation and cultured for 21 days at 37° C. and 5% CO₂ according to the reference protocol (Franceschetti et al., 2009). On day 0, cells were supplemented with 1,000 U/mL IFN-γ, followed by the addition of 50 ng/mL anti-CD3 antibody and 500 U/mL IL-2 on day 1. Fresh medium and 500 U/mL IL-2 was supplemented every 3-4 days. The phenotype of PBMCs and CIK cells was tested on days 12 and 21 and characterized by flow cytometry detecting cell surface markers CD3, CD56, CD4, CD8, CD14, and CD19, together with a cell viability dye. To explore the effect of TTFields, CIK cells were cultured for 48 hours in the presence of TTFields. CIK viability was tested by Live/Dead Aqua cell stain, and the activation state of CIK cells was evaluated by testing IFN-gamma concentration in the cell-free supernatant using a sandwich ELISA assay.

### GSC

Patient-derived CSC lines were isolated from fresh tumors using the NeuroSphere Assay and established as long-term renewing cell lines (Galli et al., (2004) Cancer Research, 64:7011-7021) in the Neural Stem Cell Biology Unit led by Dr. Galli. As a first experiment, the known effect of TTFields on GBM is tested by evaluating the viability of GSC assessed after 48 hours of exposure to TTFields. Additionally, the ability of TTFields to alter the immunogenicity of GSC is evaluated by examining the expression of relevant surface molecules such as MHC class I and NKG2D-ligand MICA/B.

### CIK and GSC Co-Culture Platform

To evaluate the anticancer activity of CIK cells against GBM, a co-culture platform with patient-derived GSC was developed. Specifically, GSCs were labeled with the CellTrace Cell Proliferation Kit and co-cultured with CIK cells at various target-to-effector ratios in 6-well flat bottom plates. After 48 hours of co-culture, apoptotic cell burden among CellTrace positive cells identified by Live/Dead Aqua cell stain or 7-amino-actinomycin D (7AAD) was used as the primary measure of CIK killing activity. CIK degranulation is used as a secondary outcome to assess CIK cell activation. To achieve this, CIK cells labeled with the CellTrace Cell Proliferation Kit are co-incubated with GSCs at an effector-to-tumor ratio of 2:1 in the presence of CD107a (Aktas et al. (2009) Cell Immunol, 254(2):149-54). After 1 hour, BrefeldinA and GolgiStop are added to each well. CIK degranulation is evaluated using flow cytometry and presented as the percentage of CD107a⁺ cells among cells labeled with CellTrace. In addition, the impact of TTFields as a pretreatment of GSCs and as a co-culture condition are tested to determine their ability to enhance the described outcomes.

### Example 4

This Example is directed to the use of TTFields as an immunotherapy to activate cytokine-induced killer (CIK) cells to increase the efficacy of CIK cell-based cancer therapies. The concurrent therapy of TTFields with CIK cells provides a synergistic effect over either treatment alone.

Human subjects are treated with TTFields by application of an OPTUNE® device (Novocure Limited, St. Helier, Jersey) to the skin of the subject, with placement of the pair of arrays left and right (LR) of the tumor and/or anterior and posterior (AP) to the tumor. Each subject is then chronically treated with TTFields at 150-200 kHz; the device is worn at least about 80% of the time, with minor breaks in between sessions and slight adjustments to the placements of the arrays, to allow the cells and skin to recover.

A composition containing CIK cells is prepared. In one instance, CIK cells are prepared as described in Example 3 using PBMCs obtained from an autologous and/or allogeneic source(s). The CIK cells are then cultured prior to implantation/injection for adoptive cell transfer therapy.

Two weeks after application of the TTFields has commenced, approximately 1-10 billion CIK cells are implanted or injected in the human subjects about 1-3 times a week for 6-8 weeks.

Following the concurrent therapy treatment, the effects of said concurrent therapy on the tumor(s) is evaluated.

### Non-Limiting Illustrative Embodiments of the Inventive Concept(s)

Illustrative embodiment 1. A method of reducing viability of cancer cells, the method comprising the steps of: (1) applying an alternating electric field to the cancer cells for a period of time; and (2) administering at least one composition to the cancer cells, wherein the at least one composition comprises at least one killer cell. The method may be an in vitro method or an in vivo method.

Illustrative embodiment 2. A method of treating cancer in a subject, the method comprising the steps of: (1) applying an alternating electric field to a target region of the subject for a period of time; and (2) administering at least one composition to the subject, wherein the at least one composition comprises at least one killer cell.

Illustrative embodiment 3. A method of reducing a volume of a tumor and/or preventing an increase of volume of the tumor, wherein the tumor is present in a body of a living subject and includes a plurality of cancer cells, the method comprising the steps of: (1) applying an alternating electric field to a target region of the subject for a period of time; and (2) administering at least one composition to the subject, wherein the at least one composition comprises at least one killer cell.

Illustrative embodiment 4. A method, comprising the steps of: (1) applying an alternating electric field to a target region of the subject for a period of time; and (2) administering at least one composition to the subject, wherein the at least one composition comprises at least one killer cell; and wherein administration of the alternating electric field increases the efficacy and/or cytotoxicity of the killer cells against cancer cells in the subject when compared to the administration of killer cells to the subject in the absence of alternating electric field application.

Illustrative embodiment 4A. The method of any of illustrative embodiments 1-4, wherein the at least one killer cell comprises at least one natural killer (NK) cell.

Illustrative embodiment 4B. The method of any of illustrative embodiments 1-4A, wherein the at least one killer cell comprises at least one cytokine-induced killer (CIK) cell.

Illustrative embodiment 4C. The method of any of illustrative embodiments 1-4B, wherein the at least one killer cell comprises at least one NK cell and at least one CIK cell.

Illustrative embodiment 5. The method of any of illustrative embodiments 1-4, wherein at least one of: the alternating electric field is applied at a frequency in a range of from about 50 kHz to about 1 MHz; the alternating electric field has a field strength of at least about 1 V/cm in at least a portion of the cancer cells/target region of the subject; the alternating electric field is induced by an applied voltage of at least 50 V RMS or at least 50 V p2p; and the period of time that the alternating electric field is applied is at least about 50% of at least about a 24 consecutive hour time period.

Illustrative embodiment 6. The method of any of illustrative embodiments 1-5, wherein the alternating electric field is applied to the cancer cells/target region of the subject for a period of time sufficient to reduce MHC Class I expression in the cancer cells compared to control cancer cells that have not been exposed to alternating electric fields.

Illustrative embodiment 7. The method of any one of illustrative embodiments 1-6, wherein the at least one killer cell comprises at least one autologous killer cell.

Illustrative embodiment 8. The method of any one of illustrative embodiments 1-7, wherein the at least one killer cell comprises at least one allogeneic killer cell.

Illustrative embodiment 9. The method of any one of illustrative embodiments 1-8, wherein at least one of: the at least one killer cell is derived from ex vivo differentiation of pluripotent stem cells; the at least one killer cell is derived from an interleukin-2 (IL-2) dependent cell line; and/or the at least one killer cell comprises a chimeric antigen receptor-engineered killer cell.

Illustrative embodiment 9A. The method of any one of illustrative embodiments 1-8, wherein at least one of: the at least one killer cell is at least one NK cell derived from ex vivo differentiation of pluripotent stem cells; the at least one killer cell is at least one NK derived from an interleukin-2 (IL-2) dependent cell line; and/or the at least one killer cell comprises a chimeric antigen receptor-engineered NK (CAR-NK) cell.

Illustrative embodiment 9B. The method of any one of illustrative embodiments 1-9A, wherein the at least one killer cell is at least one CIK cell derived from peripheral blood mononuclear cells (PBMCs) cultured in the presence of at least one of interleukin-2 (IL-2), anti-CD3 antibody, and interferon-gamma (IFN-γ).

Illustrative embodiment 10. The method of any one of illustrative embodiments 1-9, wherein the composition further comprises at least one substance selected from the group consisting of an immune checkpoint inhibitor, interleukin-1 (IL-1), IL-1R, IL-2, IL-11, IL-15, IL-17, IL-18, IL-21, lirilumab, monalizumab, and combinations thereof.

Illustrative embodiment 11. The method of any one of illustrative embodiments 1-10, wherein the composition further comprises at least one substance selected from the group consisting of an anti-PD-1 therapeutic agent, an anti-PD-L1 therapeutic agent, a chemotherapeutic agent, Paclitaxel, Docetaxel, Ifosamide, Etoposide, Gemcitabine, Lomustine, nab Paclitaxel, temozolomide, Carboplatin, a TKI inhibitor, an mTOR inhibitor, an Akt inhibitor, a PI3K inhibitor, a PARP inhibitor, an FGF inhibitor, an anti-LAB3 agent, an anti-CTLA-4 therapeutics, an aromatase inhibitor, Denosumab, pembrolizumab, and combinations thereof.

Illustrative embodiment 12. The method of any one of illustrative embodiments 1-11, wherein the method further comprises the step of: (3) administering at least one additional composition to the cancer cells/subject, wherein the at least one additional composition comprises a substance selected from the group consisting of an immune checkpoint inhibitor, interleukin-1 (IL-1), IL-1R, IL-2, IL-11, IL-15, IL-17, IL-18, IL-21, lirilumab, monalizumab, and combinations thereof.

Illustrative embodiment 13. The method of any one of illustrative embodiments 1-12, wherein the method further comprises the step of: (3) administering at least one additional composition to the cancer cells/subject, wherein the at least one additional composition comprises a substance selected from the group consisting of an anti-PD-1 therapeutic agent, an anti-PD-L1 therapeutic agent, a chemotherapeutic agent, Paclitaxel, Docetaxel, Ifosamide, Etoposide, Gemcitabine, Lomustine, nab Paclitaxel, temozolomide, Carboplatin, a TKI inhibitor, an mTOR inhibitor, an Akt inhibitor, a PI3K inhibitor, a PARP inhibitor, an FGF inhibitor, an anti-LAB3 agent, an anti-CTLA-4 therapeutics, an aromatase inhibitor, Denosumab, pembrolizumab, and combinations thereof.

Illustrative embodiment 14. The method of any one of illustrative embodiments 1-13, wherein steps (1) and (2) are performed wholly or partially sequentially, and wherein the at least one composition is administered after the application of the alternating electric field has begun.

Illustrative embodiment 15. The method of illustrative embodiment 14, wherein the at least one composition is administered before the period of time the alternating electric field is applied has elapsed.

Illustrative embodiment 16. The method of illustrative embodiment 14 or 15, wherein the at least one composition is administered after the period of time has elapsed.

Illustrative embodiment 17. The method of any one of illustrative embodiments 1-16, wherein steps (1) and (2) are repeated one or more times.

Illustrative embodiment 18. The method of any one of illustrative embodiments 1-17, wherein the cancer cells/cancer/tumor is in the form of at least one solid tumor.

Illustrative embodiment 19. The method of any one of illustrative embodiments 1-18, wherein the cancers/cancer cells are selected from the group consisting of hepatocellular carcinoma/carcinoma cells, glioblastoma/glioblastoma cells, pleural mesothelioma/mesothelioma cells, differentiated thyroid cancer/cancer cells, advanced renal cell carcinoma/carcinoma cells, ovarian cancer/cancer cells, pancreatic cancer/cancer cells, lung cancer/cancer cells, breast cancer/cancer cells, and combinations thereof.

Illustrative embodiment 20. A kit for reducing viability of cancer cells, the kit comprising: components configured for isolating and culturing at least one natural killer cell, or a precursor thereof, from a subject; and, optionally, a field generating device configured to apply an alternating electric field to the cancer cells for a period of time.

While the attached disclosures describe the inventive concept(s) in conjunction with the specific experimentation, results, and language set forth hereinafter, it is evident that many alternatives, modifications, and variations will be apparent to those skilled in the art. Accordingly, it is intended to embrace all such alternatives, modifications, and variations that fall within the spirit and broad scope of the present disclosure.

What is claimed is:

1. A method of treating cancer in a subject, the method comprising the steps of:
(1) applying an alternating electric field to a target region of the subject for a period of time; and
(2) administering at least one composition to the subject, wherein the at least one composition comprises at least one natural killer (NK) cell.

2. The method of claim 1, wherein at least one of:
the alternating electric field is applied at a frequency in a range of from about 50 kHz to about 1 MHz;
the alternating electric field has a field strength of at least about 1 V/cm in at least a portion of the target region of the subject;
the alternating electric field is induced by an applied voltage of at least 50 V p2p; and
the period of time that the alternating electric field is applied is at least about 50% of at least about a 24 consecutive hour time period.

3. The method of claim 1, wherein the alternating electric field is applied to the target region of the subject for a period of time sufficient to reduce MHC Class I expression in cancer cells in the target region compared to control cancer cells that have not been exposed to alternating electric fields.

4. The method of claim 1, wherein the at least one NK cell comprises at least one autologous NK cell.

5. The method of claim 1, wherein the at least one NK cell comprises at least one allogeneic NK cell.

6. The method of claim 1, wherein at least one of:
the at least one NK cell is derived from ex vivo differentiation of pluripotent stem cells;
the at least one NK cell is derived from an interleukin-2 (IL-2) dependent cell line; and/or
the at least one NK cell comprises a chimeric antigen receptor-engineered NK (CAR-NK) cell.

7. The method of claim 1, wherein steps (1) and (2) are performed wholly or partially sequentially, and wherein the at least one composition is administered after the application of the alternating electric field has begun.

8. The method of claim 1, wherein steps (1) and (2) are repeated one or more times.

9. The method of claim 1, wherein the cancer is selected from the group consisting of hepatocellular carcinoma, glioblastoma, pleural mesothelioma, differentiated thyroid cancer, advanced renal cell carcinoma, ovarian cancer, pancreatic cancer, lung cancer cell, breast cancer, and combinations thereof.

10. A method of reducing a volume of a tumor and/or preventing an increase of volume of the tumor, wherein the tumor is present in a body of a living subject and includes a plurality of cancer cells, the method comprising the steps of:
(1) applying an alternating electric field to a target region of the subject for a period of time; and
(2) administering at least one composition to the subject, wherein the at least one composition comprises at least one natural killer (NK) cell.

11. The method of claim 10, wherein at least one of:
the alternating electric field is applied at a frequency in a range of from about 50 kHz to about 1 MHz;
the alternating electric field has a field strength of at least about 1 V/cm in at least a portion of the target region of the subject;
the alternating electric field is induced by an applied voltage of at least 50 V p2p; and
the period of time that the alternating electric field is applied is at least about 50% of at least about a 24 consecutive hour time period.

12. The method of claim 10, wherein the alternating electric field is applied to the target region of the subject for a period of time sufficient to reduce MHC Class I expression in the cancer cells compared to control cancer cells that have not been exposed to alternating electric fields.

13. The method of claim 10, wherein the at least one NK cell comprises at least one autologous NK cell.

14. The method of claim 10, wherein the at least one NK cell comprises at least one allogeneic NK cell.

15. The method of claim 10, wherein at least one of:
the at least one NK cell is derived from ex vivo differentiation of pluripotent stem cells;
the at least one NK cell is derived from an interleukin-2 (IL-2) dependent cell line; and/or
the at least one NK cell comprises a chimeric antigen receptor-engineered NK (CAR-NK) cell.

16. The method of claim 10, wherein steps (1) and (2) are performed wholly or partially sequentially, and wherein the at least one composition is administered after the application of the alternating electric field has begun.

17. The method of claim 10, wherein steps (1) and (2) are repeated one or more times.

18. The method of claim 10, wherein the cancer is selected from the group consisting of hepatocellular carcinoma, glioblastoma, pleural mesothelioma, differentiated thyroid cancer, advanced renal cell carcinoma, ovarian cancer, pancreatic cancer, lung cancer cell, breast cancer, and combinations thereof.

19. A method, comprising the steps of:
(1) applying an alternating electric field to a target region of the subject for a period of time; and
(2) administering at least one composition to the subject, wherein the at least one composition comprises at least one natural killer (NK) cell; and
wherein administration of the alternating electric field increases the cytotoxicity of the NK cells against cancer cells in the subject when compared to the administration of NK cells to the subject in the absence of alternating electric field application.

20. The method of claim 19, wherein at least one of:
the alternating electric field is applied at a frequency in a range of from about 50 kHz to about 1 MHz;
the alternating electric field has a field strength of at least about 1 V/cm in at least a portion of the target region of the subject;
the alternating electric field is induced by an applied voltage of at least 50 V p2p; and
the period of time that the alternating electric field is applied is at least about 50% of at least about a 24 consecutive hour time period.
